(19) United States
(12) Patent Application Publication
(10) Pub. No.: US 2026/0044516 A1
(43) Pub. Date: Feb. 12, 2026

(54) FEDERATION OF SCORING SYSTEMS

(71) Applicant: NATIONAL UNIVERSITY OF SINGAPORE, Singapore (SG)

(72) Inventors: Nan LIU, Singapore (SG); Siqi LI, Singapore (SG); Marcus Eng Hock ONG, Singapore (SG)

(73) Assignee: NATIONAL UNIVERSITY OF SINGAPORE, Singapore (SG)

(21) Appl. No.: 19/101,925

(22) PCT Filed: Aug. 18, 2023
(86) PCT No.: PCT/SG2023/050574

§ 371 Date: Feb. 7, 2025

(30) Foreign Application Priority Data
Aug. 19, 2022 (SG) 10202250794A

(51) Int. Cl. G06F 16/2457; G06N 3/098

(52) U.S. Cl. CPC G06F 16/24578; G06N 3/098

(57) ABSTRACT

Systems and methods for federated scoring by a plurality of nodes, wherein each node comprises sensitive data based on which a first set of scoring model coefficients generated. The first set of scoring model coefficients are broadcast to rest of the nodes and at least one node generates a federated scoring model based on the received contributory intermediate statistics and its respective first set of scoring model coefficients.

### TECHNICAL FIELD

This disclosure generally relates to methods and systems for the federation of scoring systems or scoring models.

### BACKGROUND

This background description is provided for the purpose of generally presenting the context of the disclosure. Contents of this background section are neither expressly nor impliedly admitted as prior art against the present disclosure.

Cross-institutional partnerships in research, including healthcare research, have been increasingly popular. With the computerization of healthcare processes, a large volume of healthcare data is being generated by interactions of individuals with healthcare systems. The volume of data being generated reflective of healthcare interactions or outcomes will continue to increase as healthcare service providers continue to further automate or computerize greater parts of their services. The increasing amount of data provides a great opportunity to develop and implement computational constructs such as Machine Learning (ML) models in healthcare systems. The increasing volume of data regarding healthcare interactions presents opportunities to develop and train ML models that are more robust and accurate. As more data is available to ML models, the ML models may be trained to more accurately model diverse real-world situations.

However, data held by individual institutions are subject to constraints including privacy constraints that limit the sharing or transmission of data to other entities or institutions. The inability to share data presents a significant hurdle in the development of ML models by an individual institution or data holder and it is desirable to provide frameworks, systems and methods that address the problem or at least provide an alternative to existing solutions.

### SUMMARY

In one embodiment, the present disclosure provides a system for federated scoring, the system comprising:
a plurality of nodes,
a communication network enabling communication between the plurality of nodes, each node comprising at least one processor and a memory, the memory of each node comprising sensitive data and program code,
   the sensitive data comprises a plurality of records;
   wherein the program code is executable by the respective processor of each of the plurality of nodes to:
   generate a first set of scoring model coefficients based on sensitive data accessible to the respective nodes;
   broadcast the first set of scoring model coefficients to rest of the nodes;
and wherein at least one of the nodes is configured to:
   receive contributory intermediate statistics from the rest of the nodes;
and generate a federated scoring model based on the received contributory intermediate statistics and its respective first set of scoring model coefficients.

In another embodiment, the present disclosure provides a method for federated scoring comprising:
providing a plurality of nodes, each node comprising:
   at least one processor and a memory,
   the memory of each node comprising sensitive data and program code the sensitive data comprises a plurality of records;
providing a communication network enabling communication between the plurality of nodes,
executing the program code by the respective processor of each of the plurality of nodes to:
   generate a first set of scoring model coefficients based on sensitive data accessible to the respective nodes;
   broadcast the first set of scoring model coefficients to rest of the nodes;
executing the program code by at least one of the nodes to:
   receive contributory intermediate statistics from the rest of the nodes; and
   generate a federated scoring model based on the received contributory intermediate statistics and its respective first set of scoring model coefficients.

One or more non-transitory computer-readable storage media are also disclosed, the storage medium or media storing instructions that when executed by one or more processors cause the one or more processors to perform the method described above.

### BRIEF DESCRIPTION OF THE DRAWINGS

Some embodiments of systems and methods for privacy-preserving federated execution of scoring models are described, by way of non-limiting example only, with reference to the accompanying drawings in which:

FIG. 1 illustrates a schematic block diagram of a system for federated scoring;

FIG. 2 illustrates data partitioning for an experiment using the system for federated scoring;

FIG. 3 illustrates the performance of various scoring models at various nodes in an experiment;

FIG. 4 illustrates the combined performance of various scoring models including pooled and federated scoring models;

FIG. 5 illustrates parsimony plots generated during performance evaluation of scoring models in an experiment; and

FIG. 6 is a block diagram of a system for federated scoring.

### DETAILED DESCRIPTION

Federated learning, also known as distributed learning or distributed algorithms, can address the problems associated with limitations on data sharing by collectively training algorithms without exchanging data. In the context of healthcare data, embodiments of federated scoring systems and methods disclosed herein can safeguard patient privacy by distributing the model training to the data owners and aggregating the results across the various data owners/sites. Federated learning breaks down data silos and allows for faster development of much-needed scoring systems/models for the analysis of healthcare data and decision-making based on healthcare data. In addition, federation systems disclosed herein enable the federation of interpretable models that are preferred in clinical research.

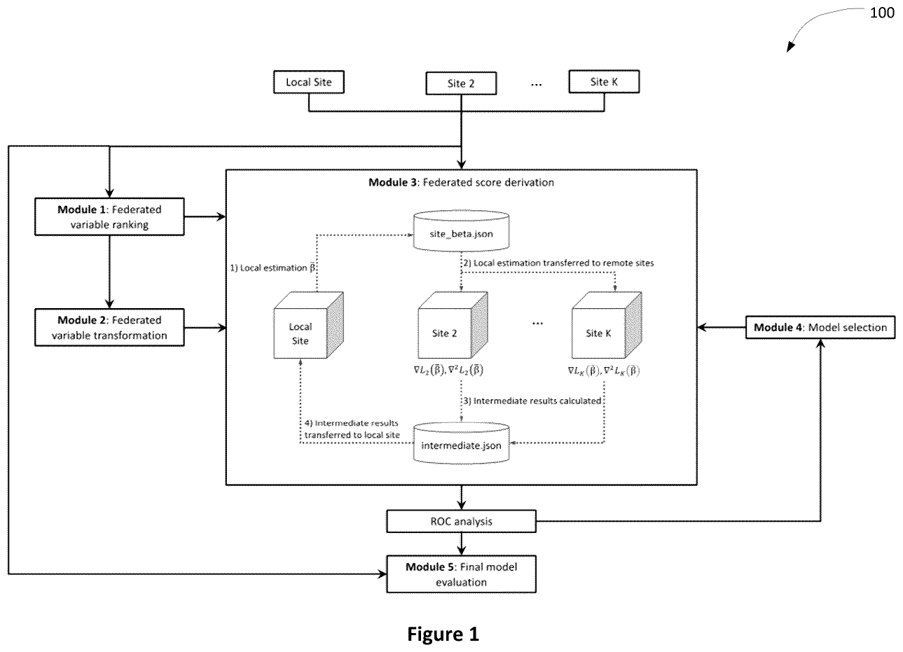

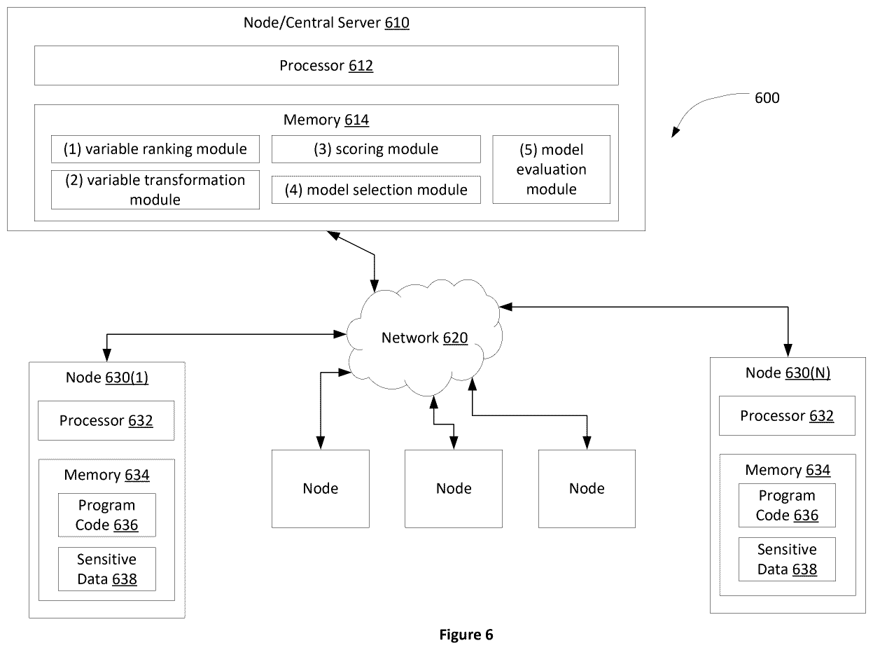

FIGS. 1 and 6 show system architectures for federated scoring. Firstly, with reference to FIG. 6, a system 600 is used for federated scoring. The system 600 includes a plurality of nodes 630 that communicate between each other over a communication network 620. As shown for nodes 630(1) and 630(N) (indicating that there are N nodes in this system or network 600), each node comprises at least one processor 632 and a memory 634. The memory 634 comprises sensitive data 638 and program code 636. The sensitive data 638 can include a plurality of records such as patient data records.

The program code 636 is executable by the respective processor 632 of each node 630 to generate a first set of scoring model coefficients based on sensitive data accessible to the respective nodes. In general, there will be two scenarios: a node 630 has access only to the sensitive data 638 stored in its own memory 634; a node from a group of affiliated nodes (e.g. a group of clinics operated by a particular company) has access to the sensitive data stored on all the affiliated nodes. Regarding coefficients, the example model used involves logistic regression, which is logit(Pr(Y=1|X))=β₀+β₁X₁+β₂X₂+ . . . . Here p is the probability of response Y=1 (Y can only be either 0 and 1), and X₁, X₂ . . . are the predictors, such as gender, age etc. Since it is possible to have more than one predictor (X), the visualization can be multi-dimensional instead of being 2D. logit(Pr(Y=1|X)) would be the y axis, but there can be more than one x axis). So, the coefficients are the betas.

The instructions stored in the memory 634 also cause the processor(s) 632 to broadcast the first set of scoring model coefficients to rest of the nodes 630.

Each node 630 is also configured to generate a node specific scoring model based on the first set of scoring model coefficients.

At least one of the nodes 630 is configured to receive contributory intermediate model statistics (also referred to as contributory intermediate statistics and others) from the rest of the nodes 630 and discussed below. That node or nodes then generate a federated scoring model based on these intermediate statistics and its respective data. Intermediate statistics are, for example, the components of Equation (5) for any particular node.

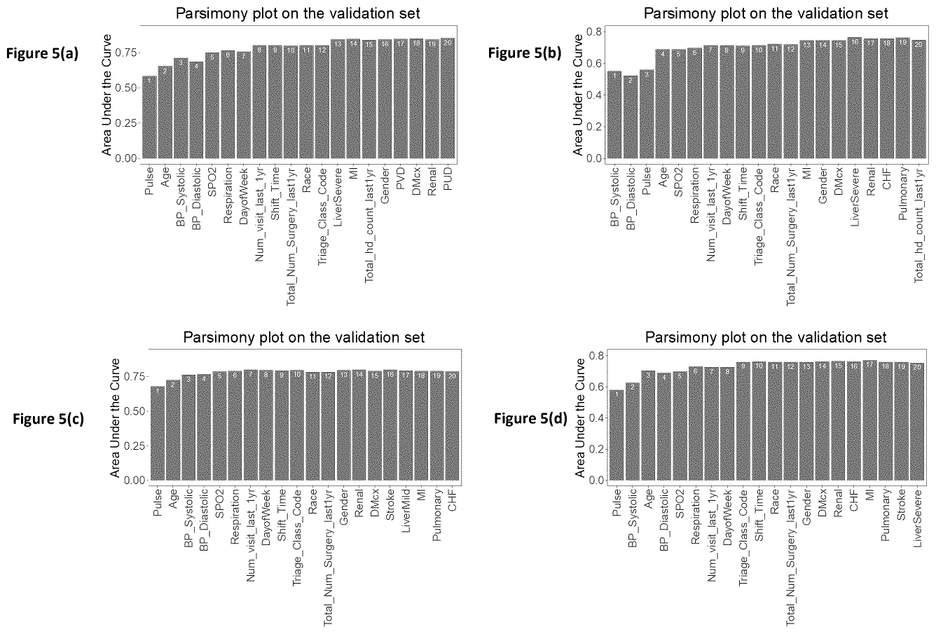

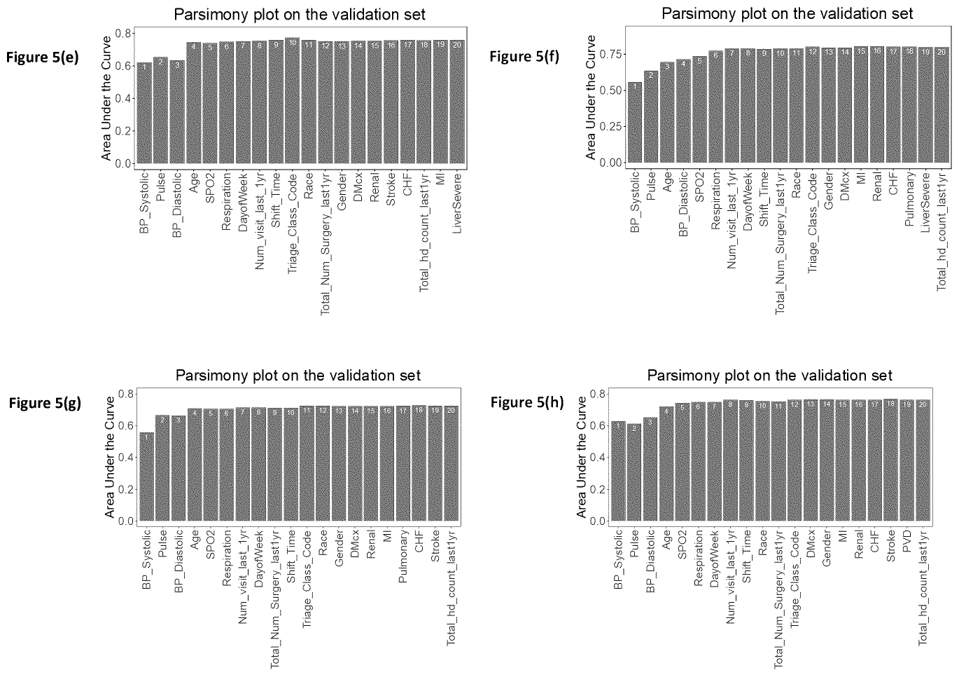

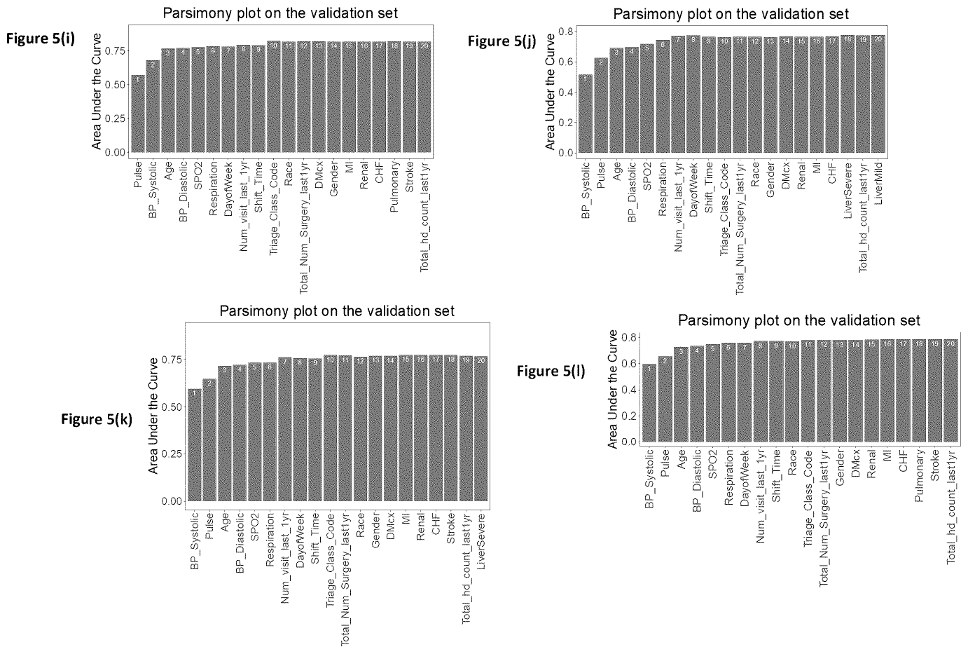

Each of the plurality of nodes 630 is configured to transmit its node specific scoring model and scoring model performance data to the central server 610. Since at least one of the nodes 630 generates a federated scoring model, the central server 610 is also configured to receive the federated model from the node or nodes 630 that create such a federated scoring model. To ensure a consistent model is implemented across some or all of the nodes 630, the central server 630 is configured to transmit the federated model to at least a subset of the plurality of nodes 630. Where more than one federated scoring model is received at the central server 610, the central server 610 may select a particular federated scoring model to be deployed on all of the nodes 630. The model selection may be conducted via parsimony plot as shown in FIG. 5. In some embodiments, this step involves user selection and in other embodiments selection is automatic based on a general criterion that adding new variables in the model would not result in increase in model performance above a predetermined threshold (increase of AUC values). That predetermined threshold may be set by a user. Variables may also be added based on domain knowledge—this can either be accessed using natural language processing over publications in the relevant domain, to identify variables most frequently mentioned, or may be based on a users domain knowledge (i.e. user added variables). These additional variables may not necessarily completely align with the parsimony plots. For example, suppose in the parsimony plot the model performance is already 0.85 using the first five variables (which is high enough to stop adding new variables), a further variable may be included based on domain knowledge or domain publications.

Some embodiments federate scoring systems across various nodes of computer systems. Scoring systems can be classification models that comprise the definition of a series of computations on input data, the computations being executed to make a prediction based on the input data. The scoring models can be implemented using various types of model, such as linear classification models, logistic regression models, and clinical decision support models. The series of computations include computations such as addition, subtraction, multiplication etc., The models are used to assess the risk of numerous serious medical conditions since they provide efficient and interpretable predictions.

Table 1 shows an example of a scoring system.

TABLE 1
A scoring system for sleep apnoea screening
Patient screens positive for obstructive sleep apnoea if Score >1

| | | | |
|---|---|---|---|
| 1. age ≥60 | 4 | points | . . . |
| 2. hypertension | 4 | points | + . . . |
| 3. body mass index ≥30 | 2 | points | + . . . |
| 4. body mass index ≥40 | 2 | points | + . . . |
| 5. female | −6 | points | + . . . |

| | | |
|---|---|---|
| Add points from row 1-6 | Score | = . . . |

A doctor can easily determine whether a patient screens positive for obstructive sleep apnoea by adding points for the patient's age, whether they have diabetes, body mass index, and sex. If the score is above a threshold, the patient would be recommended to a clinic for a diagnostic test.

Traditional scoring systems have largely been developed on single-source data. Consequently, training or sample data sets are often small or not representative—e.g. data taken from an affluent community will likely have a lower rate of adverse outcomes than data from a poor community. Although it is possible to develop scoring systems on pooled data, the pooling process is time-consuming and difficult to achieve due to privacy reasons. The framework shown in FIG. 6 is for building scoring systems in a federated manner to address such difficulties.

The disclosed systems and method (also referred to as FedScore) provide an approach for building federated scoring systems executable across multiple computer system nodes provided at various locations. The embodiments improve robustness and remove biases from medical research, particularly research in contexts with relatively small sample sizes. FIGS. 1 and 6 show the overall architecture of a system according to the embodiments.

### FedScore Framework

When implementing clinical models and other models where data privacy is to be maintained (e.g. financial records), users usually consider the degree of parsimony as a key characteristic of the model. A model is considered parsimonious when it is sparse (i.e., it uses the least amount of variables possible) and has good prediction accuracy. FIG. 6 illustrates a block diagram of a FedScore system/framework 600. The FedScore system of some embodiments may comprise a central server 610 and a plurality of nodes 630. Alternatively, a specific node of the plurality of nodes of the FedScore system may perform the functions of the central server and the node or system/framework 600 thereby not requiring a designated central server. As discussed above, each node 630 comprises at least one processor 632 and a memory 634. Memory 634 comprises program code 636 and sensitive data 638. The various nodes are in communication with each other over a communication network 620. The sensitive data is accessible only to the respective nodes.

To incorporate the privacy requirement while achieving good parsimony and interpretability, the FedScore framework consists of five modules: (1) federated variable ranking module; (2) federated variable transformation module; (3) federated score derivation module/scoring module; (4) model selection module and (5) model evaluation module. Some or all of these modules may be provided in a particular node, distributed across nodes or on the central server depending on the architecture of the specific embodiment. In the embodiment shown in FIG. 6, these modules are stored in memory 614.

### (1) Federated Variable Ranking Module

To construct a global model across several sites or nodes, some embodiments may pre-identify a set of unified variables as candidate variables for ranking to be performed independently across the various nodes 630. For example: suppose sites A and B both use 0%, 25%, 50%, 75% and 100% to cut their variables. Due to data heterogeneity, their cut off for variable age may be different: A: (,24], (24, 49], (49, 62], (62,) years; B: (,24], (24, 52], (52, 67], (67,) years. This suggests that site B has a relatively older population. In this case, federation cannot be conducted, as these categorical variables have different meanings and are not unified. Instead, when federating, the two sites specify cuts that are sensible for both sites—e.g. (,24], (24, 50], (50, 60], (60,) years, with federation then being able to be conducted. This unification may result from collecting data from all contributing sites (e.g. the ages of all patients) and segregating based on statistical paramteres such as percentiles as mentioned above. In some embodiments, random forests may be utilized to perform variable ranking. In the FedScore framework, variable ranking is first performed at each local site/node 630. Each node 630 then transmits the variable rankings to a central server 610 that generates a global variable ranking. The global variable ranking may be generated by ordering variables by their averaged ranks at each site.

The random forests of some embodiments may comprise a collection of randomized classification and regression trees. One importance measurement of a given variable in random forests is the increase in mean of a tree's error when the observed values of a particular variable are randomly permuted in out-of-bag samples. More specifically, the importance of a variable may be quantified based on the mean square error for regression and misclassification rate for classification. In classification tasks, Gini index is defined for each node θ for a decision tree Θ as:

$$Gini ( θ ) = {\sum }_{r = 1}^{R} {p}_{r} \times ( 1 - {p}_{r} ) \qquad ( 1 )$$

where pᵣ is the fraction of training samples in the r^{th} class at the node, and R=2 for a binary classification task. The importance of a variable Xₘ is the weighted total impurity decrease w(θ)ΔGini(θ) for all nodes. When averaged over all trees this metric is calculated as:

$$Imp ( {X}_{m} ) = \frac{1}{{N}_{θ}} {\sum }_{Θ} {\sum }_{θ ∈ Θ : v ( θ ) = {X}_{m}} w ( θ ) Δ Gini ( θ ) \qquad ( 2 )$$

where w(θ) is the proportion N_{θ}/N of samples reaching node θ, ΔGini(θ) is the impurity decrease after the split of node θ, and v(θ) is the variable used in the split. In the FedScore framework, variable ranking is performed first at each local site/node, and then a global variable ranking is created by rearranging variables by their averaged ranks at each site.

In some embodiments, the variable ranking at each node may be determined based on a model parsimony statistic such as a model parsimony plot which demonstrates the relevance of each variable or a combination of variables to the performance of the scoring model. In some embodiments, the area under curve statistic of various models may be used to evaluate the variable ranking. The federated scoring model of some embodiments may take into account variables in the global variable rank that are above a predefined threshold such as an importance threshold. For example, suppose a random is used forest for importance measurement. After scaling importance values to 0 and 1, the following variable importance values may result: var1: 0.8, var2: 0.5, var3: 0.3, var4: 0.2, var5: 0.15, var6: 0.08 . . . . The threshold can be empirically set to be 0.1, resulting in selection of only the first five variables. By doing so, the federated scoring model reduces the number of variables incorporated in the model and serves as a model that is more interpretable while retaining its accuracy in clinical environments—i.e. the model becomes sparse without significant loss in accuracy.

### (2) Federated Variable Transformation Module

The FedScore framework also transforms continuous variables into categorical/discrete variables after the global variable ranking is determined. For example, the age of a person may be banded—e.g. 0 to 20 years old, 20 to 30 years old, 30 to 45 years old, 45 to 60 years old, and 60 years old and older. The maximum number of categories for such transformation may be pre-determined (for example, 5), and if the maximum value for a particular variable is surpassed, categories may be combined so that the maximum requirement is met. A global cutoff/discrete bucket for each continuous variable is calculated by averaging the k values acquired at each site. k values are used to cut a continuous variable into several categorical variables. For example, suppose there is only one k value of 50% for variable age, and the 50% cutoff for age is 50 years old, the age variable would be transformed from a continuous variable to two categorical variables: age (<=50) (true of false) and age (>=50) (true or false). After defining the discrete buckets/cutoffs for each variable, the defined cutoffs are transmitted to the plurality of nodes to enable the nodes to process the continuous variable data in a unified and standardized discrete manner. In some embodiments, quantiles of continuous variables were set to be 0%, k₁%, k₂%, k₃%, k₄% and 100%, where the value of k₁, k₂, k₃, k₄ was set to 5, 20, 80 and 95. The federated variable transformation by providing the standardized discrete buckets for the continuous variables improves the accuracy of the federation as the various nodes observe a common set of discrete buckets when providing input to their respective scoring models.

### (3) Federated Score Derivation Module

The score derivation process could be flexibly adjusted for different clinical modelling purposes by incorporating a suitable ML model depending on the clinical need and the context. For instance, a logistic regression model may only support binary outcomes. By switching the logistic regression in Module 3 to other suitable models, the frameworks can be expanded to support survival outcomes and ordinal outcomes etc (i.e. non-binary outcomes). A step of the score derivation process includes the generation of a first set of scoring model coefficients based on sensitive data. The scoring model coefficients include the various parameters of the scoring model. For example, in embodiments where the scoring model is a linear model, the coefficients are the linear parameters and intercepts, etc. Each node generates its own first set of scoring model coefficients because each node has access to different sensitive data. In general, the data will be non-overlapping. The first set of scoring model coefficients are used by each node to define a node-specific scoring model. The node-specific scoring models serve as candidate models for comparison with the federated scoring model.

The first set of scoring model coefficients may be broadcast to the rest of the nodes and each node may generate a set of contributory/intermediate scoring model coefficients based on the received first set of scoring model coefficients and the sensitive data accessible to each respective node. The contributory scoring model coefficients may subsequently be transmitted/broadcast to the rest of the nodes. One or more nodes may generate a federated scoring model based on its first set of scoring mode coefficients and the contributory scoring mode coefficients received from the rest of the nodes. An example of federated scoring model generation is described with reference to an ODAL2 algorithm.

As another example, logistic regression is a common choice for modelling binary outcomes. Federated logistic regression may be implemented by some embodiments calling for multiple iterations of logistic regression or logistic regression over one iteration (one-shot approach).

FedScore is a privacy-preserving framework to provide unified and robust scoring systems across multiple sites without the need for sharing sensitive data, such sensitive medical data or other personal information. FedScore was tested using models for clinical scoring for 30-day mortality prediction utilizing emergency department (ED) data from Singapore General Hospital (SGH) and a simulation of 10 nodes/sites that did not exchange sensitive patient data during the experiment. FedScore's robustness and generalizability were established by achieving a high average area under the curve (AUC) on the testing data of each site with the smallest variance when compared to baseline scores.

Experiments were performed using an ODAL2, a one-shot privacy preserving distributed algorithm as disclosed in R. Duan et al., “Learning from electronic health records across multiple sites: A communication-efficient and privacy-preserving distributed algorithm,” J. Am. Med. Inform. Assoc., vol. 27, no. 3, pp. 376-385, December 2019, doi: 10.1093/jamia/ocz199 to execute federated logistic regression. Embodiments utilized information from the local site/node with the first-order (ODAL1) (first set of scoring model coefficients) and second-order (ODAL2) gradients (contributory scoring model coefficients) of the likelihood function from remotes sites to construct an approximation of the global likelihood function that forms a part of the federated scoring model. Data from the remote sites/nodes was not accessible in the execution of the logistic regression computations. Coefficients generated at each node during the logistic regression were transmitted to the central server.

The coefficients in a global logistic regression model are generated by optimizing the likelihood function and then are rounded to obtain scores based on each variable. A scoring table was defined by the central server and the overall score is calculated by adding all the points together. The ceiling number for total score and normalization of score breakdowns could both be adjusted to fit the needs of an intended clinical application.

The coefficients of a global logistic regression model may be obtained by optimizing a global likelihood function. Let x₁, x₂, . . . xₚ₋₁ denote the p−1 predictors, y denotes a binary outcome, and the logistic regression model can be expressed as

$$log it ( Pr ( y = 1 \vert x ) ) = {x}^{T} β \qquad ( 3 )$$

where x=(1, x₁, x₂, . . . xₚ₋₁)^{T}, β is the vector of intercepts and coefficients, and logit(t)=log t/(1−t). Suppose a total of

$$N = {\sum }_{j = 1}^{K} {n}_{j}$$

identically and independently distributed (i.i.d.) observations are distributed at K sites/nodes, then the likelihood function (LLR) of global logistic regression by pooling data from all sites is

$$L ( β ) = \frac{1}{N} {\sum }_{j = 1}^{K} {\sum }_{i = 1}^{{n}_{j}} [ {Y}_{ij} {x}_{ij}^{T} β - log \{ 1 + exp ( {x}_{ij}^{T} β ) \} ] \qquad ( 4 )$$

The pooled estimator \hat{β} can be obtained by optimizing L(β).

However, when data cannot be shared computation of the pooled likelihood function is not possible. As envisaged by the embodiments, approximation of the likelihood function is performed as described herein. As an example, the ODAL2 algorithm applies the idea of Taylor expansion, proposing to use first and second order gradient of LLR (log-likelihood ratio) to perform the approximation:

$${\tilde{L}}^{2} ( β ) = {L}_{1} ( β ) + {\{ ∇ L ( \overline{β} ) - ∇ {L}_{1} ( \overline{β} ) \}}^{T} β + \frac{1}{2} {( β - \overline{β} )}^{T} \{ {∇}^{2} L ( \overline{β} ) - {∇}^{2} {L}_{1} ( \overline{β} ) \} ( β - \overline{β} ) \qquad ( 5 )$$

Here β̅ is an initial value,

$${L}_{j} ( β ) = \frac{1}{{n}_{j}} {\sum }_{i = 1}^{{n}_{j}} [ {Y}_{ij} {x}_{ij}^{T} β - log 1 + exp ( {x}_{ij}^{T} β ) ]$$

is the LLR of the j-th site (j=1 is assumed to the local site),

$$∇ L ( \overline{β} ) = {\sum }_{j = 1}^{K} {n}_{j} ∇ {L}_{j} ( \overline{β} ) / N$$

is the first gradient of LLR L(β̅) evaluated at β̅,

$$∇ {L}_{j} ( \overline{β} ) = \frac{1}{{n}_{j}} {\sum }_{i = 1}^{{n}_{j}} \{ {Y}_{ij} - {p}_{ij} ( \overline{β} ) \} {x}_{ij}$$

is first gradient of LLR of site j, where

$${p}_{ij} ( \overline{β} ) = 1 + {exp ( - {x}_{ij}^{T} \overline{β} )}^{- 1} and {∇}^{2} {L}_{j} ( \overline{β} ) = \frac{1}{{n}_{j}} {\sum }_{i = 1}^{{n}_{j}} {p}_{ij} ( \overline{β} ) \{ 1 - {p}_{ij} ( \overline{β} ) \} {x}_{ij} {x}_{ij}^{T}$$

is the second gradient of LLR of site j.

When executing the ODAL2 algorithm, the initial value β̅ (first set of scoring model coefficients) is first obtained from the result of local logistic regression performed at each local site/node—e.g. based on data such as that represented in Table 1. Then β̅ is broadcast to the rest of the remote sites/nodes of the system. A federated scoring model is generated based on the received contributory scoring model coefficients and a node's first set of scoring model coefficients. After receiving the broadcasted first set of scoring model coefficients, a site/node may compute ∇Lⱼ(β̅), ∇²Lⱼ(β̅) to build a surrogate likelihood which forms part of a federated scoring model. The federated scoring model may be generated by one or more of the nodes or the central server depending on the configuration of the FedScore framework. In some embodiments, the federated scoring model may also take into account the global variable rank generated by the (1) federated variable ranking module. Variables that are lowly ranked may be discarded by the federated scoring model in the interest of model parsimony and interpretability.

The global beta estimator \tilde{β}² is obtained by optimizing the surrogate likelihood function. In some embodiments, two json files, ‘site_beta.json’ and ‘intermediate.json’ as illustrated in FIG. 1 are used to store values that need to be broadcast in the process. The information in these two shared files is aggregated and does not contain any patient-level information, which guarantees data privacy. Part of the process for generating the federated scoring model is illustrated in FIG. 1.

In some embodiments, the coefficients in the federated logistic regression model (federated scoring model) are rounded to get relevance scores for each variable. A scoring table is created, and an overall score is calculated by adding all the points together. A ceiling number for the total score and normalization of score breakdowns could both be adjusted to fit the needs of an intended clinical application.

### (4) Model Selection Module

The scoring models trained at each node serve as candidates that could be adopted by any or all of the rest of the nodes for obtaining the most accurate results going forward. Model evaluation and selection could be performed using parsimony plots generated by the mean AUC (area under curve) of all sites/nodes. Parsimony plots represent the performance of a scoring model as a function of the number of variables incorporated in the model. Let i denote site/node index, where i∈{1, 2, . . . . K}. A general model selection criteria could be defined by maximizing Ψₘ=Σwᵢϕᵢ(p₁, p₂, p₃, . . . pₘ), where wᵢ is the weight for site i, ϕᵢ is the scoring model's performance on ith validation set and m is a pre-defined number of total variables to include, which may be uniform across all sites. In some embodiments, weights may be defined as

$${w}_{i} = \frac{1}{K} ,$$

indicating equal weights for all sites. Yet

$${w}_{i} > \frac{1}{K}$$

may be flexibly assigned for a site i if the performance of scores on this site is considered more important than others.

Different constraints can be added for the maximization task as well. For example, the total number of variables m may not exceed an integer number N. The set of variables {p₁, p₂, . . . p_{}} may also be constrained to satisfy a predefined standard required by the system. For instance, the system of some embodiments may be configured to include in the federated scoring model a set of variables {x₁, x₂, . . . x_{q}}, where q≤m. Moreover, Ψ may be maximized using a number of d variables that are smaller than m, as long as increasing the variable numbers from d to m has an acceptable impact on the change in Ψ: |Ψₘ−Ψ_{d}|≤∈, where the size of ∈ may be decided intuitively by users.

A final selection of variables may be confirmed based on the selected federated model from among the plurality of scoring models at the respective nodes. A new scoring model may be refitted to any new data using step (2). The performance of the selected federated scoring model is validated on each testing data set of each site participating in the federated learning process. The selected federated scoring model may be transmitted to each node to allow the nodes to process new clinical data using the federated scoring model.

Notably, to maintain parsimony without loss of accuracy, variables that are interrelated may be identified—e.g. variables that have substantially the same influence on patient outcomes and vary substantially in proportion with each other (e.g. with a 5% of each other)—e.g. weight and height may vary substantially in proportion with each other (e.g. within 5%) for a particular sex. Of the interrelated variables, the system may select a best variable for the federated model (e.g. the variable of the interrelated variables, that correlates most closely with outcomes such as 30 day mortality) based on each node either capturing that variable in its sensitive data, or capturing a different variable that is interrelated with the best variable. Each node that does not capture the best variable may then substitute the relevant interrelated variable in the local model.

The resulting federated scoring model is interpretable. Unless context dictates otherwise, being interpretable means that the correlation between sensitive data and outcomes is clear and explicable from the model—e.g. age correlates well with sleep apnoea for people over 60, per Table 1. This is as opposed to machine learning models that use hidden layers to identify features in the data and thus for which correlations between data and outcomes may not be readily apparent.

### (5) Model Evaluation Module

The performance of the federated scoring model and/or the node-specific scoring models is validated using the testing data sets of each site engaged in the FedScore framework. Model evaluation may be performed by a designated node or a central server depending on the configuration of the FedScore framework. Following the Ψₘ defined in step (4), the overall average performance of a federated scoring model may be defined as:

$${M}_{1} = \frac{1}{K} \sum {μ}_{i} ( {p}_{1} , {p}_{2} , {p}_{3} , … {p}_{m} ) ,$$

where μᵢ is the scoring model's performance on ith testing set; and M₂=\sqrt{Σ(M₁−μᵢ)²/K} as a measurement of performance variation across sites. A higher M₁ value and lower M₂ value indicates a score's better performance and generalizability.

### Clinical Study Design

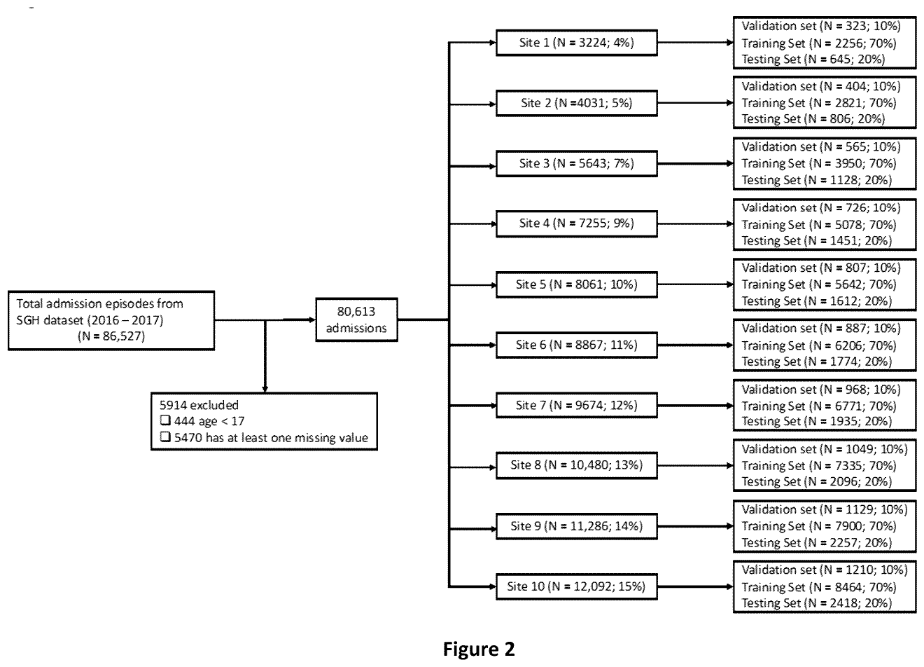

A retrospective analysis was conducted using the emergency health record data of Singapore General Hospital (SGH) extracted from the SingHealth Electronic Health Intelligence System. The initial study cohort of a total of 86527 admissions was identified by selecting ED admission at SGH data between 2016 and 2017. After excluding patients under the age of 18 and those with missing values, a total of 80613 admissions remained, which were then randomly divided into 10 simulated sites, in the proportion of 4%, 5%, 7%, 9%, 10%, 11%, 12%, 13%, 14%, and 15% respectively. Data partitioning for an experiment using the system for federated execution of an ML model is shown in FIG. 2.

The outcome of the study was 30-day mortality, which was defined as deaths that occurred within 30 days after ED admission. The candidate predictors include a total of 29 variables: demographics information, PACS triage categories, shift time, day of the week (Friday, Monday, Weekend, Midweek), vital signs, comorbidities, and previous health care usage, etc.

### Results

Analysis was performed over three groups (1) ten local scores trained independently on each site (2) one federated score trained using all sites without data sharing (3) one pooled score generated using pooled data, which is the ideal case and usually impossible in real-world applications. The models were selected based on corresponding parsimony plots, with a predefined criterion that the maximum number of variables in a model should not exceed 10 and adding more variables should cease when there is no significant improvement in AUC. The variables selected for each model are included in the footnotes of Table 2. FIG. 5 illustrates a series of parsimony plots obtained for the various scoring models. Plots (a)-(j) relate to local models generated on site 1 to site 10. Plot (k) relates to the federated scoring model generated on any one of the sites/nodes. Plot (l) relates to a scoring model generated using pooled data that would otherwise not be feasible in a real-world environment.

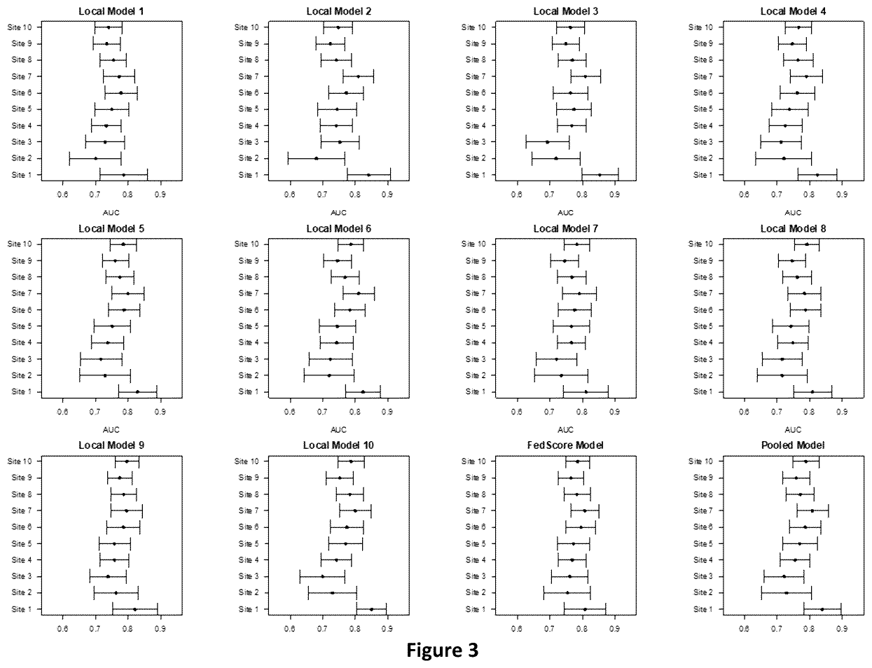

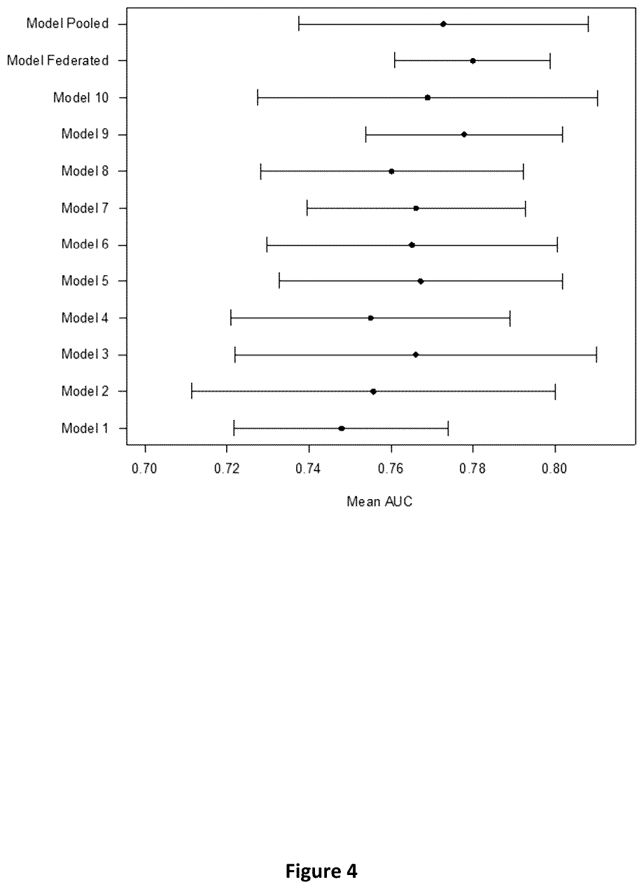

A total of twelve scoring models were obtained and tested, including ten local models generated independently on each site, one federated model developed by FedScore, and one pooled model generated based on pooled data from all the sites that would not be otherwise possible in a real-world scenario. The AUC values and confidence intervals (CI) of each model on different sites' testing data are presented in Table 2. The AUC values and 95% CIs for each score are plotted individually in FIG. 3 to better illustrate the results of Table 2 in terms of model performance variability across all sites. The mean and standard deviation (SD) of the AUC values for each model over all 10 testing sets were also computed and presented in Table 2. FIG. 4 depicts the information accordingly. As shown in both FIG. 3 and FIG. 4, the federated scoring model outperformed all local scores in terms of stability and generalizability by achieving the lowest SD. The federated score also exhibited a satisfactory average AUC for each site, indicating that the existing FedScore framework can generate global clinical scores that are trustworthy. The bottom row of Table 2 displays the averaged AUC values of all local models on each site, which are predominantly exceeded by the AUC values of the federated score. This suggests that for a single site, FedScore has the potential to yield a better scoring system than those developed locally, particularly when the sample size of the local site is inadequate.

TABLE 2
Comparison of performance of FedScore model with baseline models

| | | Testing Data | | | | | | | | | | | |
|---|---|---|---|---|---|---|---|---|---|---|---|---|---|
| | Number of | Site 1 | | Site 2 | | Site 3 | | Site 4 | | Site 5 | | Site 6 | |
| Model | variables | AUC | 95% CI | AUC | 95% CI | AUC | 95% CI | AUC | 95% CI | AUC | 95% CI | AUC | 95% CI |
| Model | 8 | 0.7867 | 0.7141- | 0.7002 | 0.6210- | 0.7295 | 0.6699- | 0.7329 | 0.6867- | 0.7501 | 0.5968- | 0.7780 | 0.7287- |
| 1 [symbol] | | | 0.8593 | | 0.7794 | | 0.789 | | 0.779 | | 0.8034 | | 0.8273 |
| Model | 7 | 0.8413 | 0.7753- | 0.6805 | 0.5937- | 0.7531 | 0.6949- | 0.742 | 0.6924- | 0.7449 | 0.6856- | 0.7722 | 0.7186- |
| 2 [symbol] | | | 0.9073 | | 0.7673 | | 0.8113 | | 0.7917 | | 0.8043 | | 0.8258 |
| Model | 7 | 0.8537 | 0.7982- | 0.719 | 0.6458- | 0.6922 | 0.6258- | 0.7677 | 0.7233- | 0.7749 | 0.7208- | 0.7634 | 0.7110- |
| 3 [symbol] | | | 0.9092 | | 0.7922 | | 0.7587 | | 0.8121 | | 0.8273 | | 0.8159 |
| Model | 9 | 0.8236 | 0.704- | 0.7208 | 0.6353- | 0.7121 | 0.6495- | 0.7256 | 0.5747- | 0.7386 | 0.6825- | 0.7618 | 0.7085- |
| 4 [symbol] | | | 0.8833 | | 0.8063 | | 0.7747 | | 0.7763 | | 0.7948 | | 0.8162 |
| Model | 10 | 0.8289 | 0.7711- | 0.7296 | 0.652- | 0.717 | 0.6531- | 0.7378 | 0.6878- | 0.7513 | 0.6960- | 0.7876 | 0.7399- |
| 5 [symbol] | | | 0.8868 | | 0.8072 | | 0.781 | | 0.7877 | | 0.8065 | | 0.8354 |
| Model | 7 | 0.8238 | 0.7703- | 0.7203 | 0.6431- | 0.7239 | 0.6572- | 0.7431 | 0.8928- | 0.7450 | 0.6893- | 0.7832 | 0.7359- |
| 6 [symbol] | | | 0.8774 | | 0.7974 | | 0.7907 | | 0.7935 | | 0.8007 | | 0.8305 |
| Model | 7 | 0.8109 | 0.7416- | 0.7347 | 0.6525- | 0.7211 | 0.6591- | 0.7662 | 0.7227- | 0.7660 | 0.7111- | 0.7760 | 0.7260- |
| 7 [symbol] | | | 0.8801 | | 0.8167 | | 0.783 | | 0.8097 | | 0.8209 | | 0.8260 |
| Model | 8 | 0.8087 | 0.7501- | 0.7157 | 0.6381- | 0.7157 | 0.6537- | 0.7478 | 0.7013- | 0.742 | 0.6850- | 0.7872 | 0.7398- |
| 8 [symbol] | | | 0.8672 | | 0.7932 | | 0.7777 | | 0.7944 | | 0.7990 | | 0.8347 |
| Model | 10 | 0.8215 | 0.7527- | 0.7631 | 0.6944- | 0.7365 | 0.6819- | 0.758 | 0.7139- | 0.7585 | 0.7099- | 0.7854 | 0.7351- |
| 9 [symbol] | | | 0.0903 | | 0.8317 | | 0.7951 | | 0.8021 | | 0.8073 | | 0.8368 |
| Model | 8 | 0.8505 | 0.8051- | 0.7296 | 0.6548- | 0.6996 | 0.6309- | 0.7423 | 0.6953- | 0.7708 | 0.7191 | 0.7744 | 0.7237- |
| 10 [symbol] | | | 0.896 | | 0.8045 | | 0.7682 | | 0.7894 | | 0.8225 | | 0.8251 |
| Model | 10 | 0.8082 | 0.7448- | 0.7537 | 0.6824- | 0.761 | 0.7058- | 0.7685 | 0.7255- | 0.7728 | 0.7239- | 0.7953 | 0.7495- |
| Federated [symbol] | | | 0.8717 | | 0.8251 | | 0.8162 | | 0.8116 | | 0.8218 | | 0.8412 |
| Model | 8 | 0.8389 | 0.7619- | 0.7288 | 0.6514- | 0.7214 | 0.6605- | 0.7550 | 0.7096- | 0.7698 | 0.7168- | 0.7864 | 0.7378- |
| Pooled [symbol] | | | 0.8959 | | 0.8063 | | 0.7823 | | 0.8005 | | 0.8228 | | 0.8350 |

| | | | | | | |
|---|---|---|---|---|---|---|
| Average AUC of all | 0.8250 | 0.7214 | 0.7203 | 0.7463 | 0.7541 | 0.7769 |
| 10 local models on | | | | | | |
| each site | | | | | | |

| | | Testing Data | | | | | | | | Mean of AUC | SD of AUC |
|---|---|---|---|---|---|---|---|---|---|---|---|
| | Number of | Site 7 | | Site 8 | | Site 9 | | Site 10 | | of each model | of each model |
| Model | variables | AUC | 95% CI | AUC | 95% CI | AUC | 95% CI | AUC | 95% CI | on all 10 sites | on all 10 sites |
| Model | 8 | 0.7725 | 0.7232- | 0.7647 | 0.7146- | 0.7345 | 0.6930- | 0.7400 | 0.6992- | 0.7479 | 0.0261 |
| 1 [symbol] | | | 0.8218 | | 0.7948 | | 0.776 | | 0.7808 | | |
| Model | 7 | 0.8092 | 0.7629- | 0.7424 | 0.6953- | 0.7234 | 0.6785- | 0.7479 | 0.7033- | 0.7657 | 0.0444 |
| 2 [symbol] | | | 0.8554 | | 0.7895 | | 0.7583 | | 0.7924 | | |
| Model | 7 | 0.8092 | 0.7636- | 0.7587 | 0.7250- | 0.7491 | 0.7077- | 0.7031 | 0.7204- | 0.7600 | 0.0442 |
| 3 [symbol] | | | 0.8547 | | 0.8125 | | 0.7906 | | 0.8057 | | |
| Model | 9 | 0.7903 | 0.7403- | 0.7646 | 0.7194- | 0.7456 | 0.7039- | 0.7682 | 0.7260- | 0.7550 | 0.0341 |
| 4 [symbol] | | | 0.8404 | | 0.8097 | | 0.7893 | | 0.8064 | | |
| Model | 10 | 0.7992 | 0.7502- | 0.7760 | 0.7327- | 0.7600 | 0.7206- | 0.7850 | 0.7454- | 0.7672 | 0.0345 |
| 5 [symbol] | | | 0.8483 | | 0.8174 | | 0.8013 | | 0.8246 | | |
| Model | 7 | 0.8103 | 0.7628- | 0.7688 | 0.7252- | 0.7453 | 0.7021- | 0.7869 | 0.7482- | 0.7051 | 0.0364 |
| 6 [symbol] | | | 0.8579 | | 0.8124 | | 0.7884 | | 0.8255 | | |
| Model | 7 | 0.7904 | 0.7380- | 0.7676 | 0.7233- | 0.7453 | 0.7023- | 0.7825 | 0.7430- | 0.7661 | 0.0267 |
| 7 [symbol] | | | 0.8420 | | 0.8119 | | 0.7883 | | 0.8211 | | |
| Model | 8 | 0.7839 | 0.7337- | 0.7621 | 0.7183- | 0.7467 | 0.7047- | 0.7917 | 0.7539- | 0.7602 | 0.0321 |
| 8 [symbol] | | | 0.8341 | | 0.8059 | | 0.7887 | | 0.8295 | | |
| Model | 10 | 0.7955 | 0.7474- | 0.7871 | 0.7487- | 0.7745 | 0.7367- | 0.7960 | 0.7597- | 0.7778 | 0.0241 |
| 9 [symbol] | | | 0.8436 | | 0.8255 | | 0.8123 | | 0.8322 | | |
| Model | 8 | 0.8001 | 0.7515- | 0.7828 | 0.7411- | 0.7521 | 0.7097- | 0.7869 | 0.7471- | 0.7689 | 0.0415 |
| 10 [symbol] | | | 0.8487 | | 0.8246 | | 0.7945 | | 0.8266 | | |
| Model | 10 | 0.8069 | 0.7641- | 0.7832 | 0.7432- | 0.7645 | 0.7255- | 0.7848 | 0.7481- | 0.7799 | 0.0190 |
| Federated [symbol] | | | 0.8498 | | 0.8232 | | 0.8026 | | 0.8215 | | |
| Model | 8 | 0.8087 | 0.7607- | 0.7710 | 0.7275- | 0.7588 | 0.7174- | 0.7886 | 0.7492- | 0.7727 | 0.0353 |
| Pooled [symbol] | | | 0.8567 | | 0.8146 | | 0.8002 | | 0.8280 | | |

| | | | | |
|---|---|---|---|---|
| Average AUC of all | 0.7961 | 0.7674 | 0.7478 | 0.7746 |
| 10 local models on | | | | |
| each site | | | | |

[symbol]

indicates data missing or illegible when filed

In Table 2, the following abbreviations apply: AUC, area under the curve; CI, confidence interval; SD, standard deviation; SBP, systolic blood pressure; DBP, diastolic blood pressure; SpO₂, oxygen saturation as measured by pulse oximetry; PACS, Patient Acuity Category Scale; ED, emergency department. Moreover: a Local model obtained via AutoScore on Site 1; variables selected in the model (in the order of ranking): pulse, age, SBP, DBP, SpO₂, respiration, day of week, ED admissions in the past year; b Local model obtained via AutoScore on Site 2; variables selected in the model (in the order of ranking): SBP, DBP, pulse, age, SpO₂, respiration, ED admissions in the past year; c Local model obtained via AutoScore on Site 3; variables selected in the model (in the order of ranking): pulse, age, SBP, DBP, SpO₂, respiration, ED admissions in the past year; d Local model obtained via AutoScore on Site 4; variables selected in the model (in the order of ranking): pulse, SBP, age, DBP, SpO₂, respiration, ED admissions in the past year, PACS triage categories; e Local model obtained via AutoScore on Site 5; variables selected in the model (in the order of ranking): SBP, pulse, DBP, age, SpO₂, respiration, day of week, ED admissions in the past year, shift time, PACS triage categories; f Local model obtained via AutoScore on Site 6; variables selected in the model (in the order of ranking): SBP, pulse, age, DBP, SpO₂, respiration, ED admissions in the past year; g Local model obtained via AutoScore on Site 7; variables selected in the model (in the order of ranking): SBP, pulse, DBP, age, SpO₂, respiration, ED admissions in the past year; h Local model obtained via AutoScore on Site 8; variables selected in the model (in the order of ranking): SBP, pulse, DBP, age, SpO₂, reparation, day of week, ED admissions in the past year; i Local model obtained via AutoScore on Site 9; variables selected in the model (in the order of ranking): pulse, SBP, age, DBP, SpO₂, respiration, day of week, ED admissions in the past year, shift time, PACS triage categories; i Local model obtained via AutoScore on Site 10; variables selected in the model (in the order of ranking): SBP, pulse, age, DBP, SpO₂, respiration, ED admissions in the past year, day of week; k Federated model obtained via FedScore; variables selected in the model (in the order of ranking): SBP, pulse, age, DBP, SpO₂, respiration, ED admissions in the past year, day of week, shift time, PACS triage categories; I Pooled model obtained via AutoScore; variables selected in the model (in the order of ranking): SBP, pulse, age, DBP, SpO₂, respiration, day of week, ED admissions in the past year.

The framework provided by the embodiments is scalable and flexible, given that each scoring model can be modified or replaced to adapt to different clinical research questions. For example, the score derivation module may be adjusted to generate ordinal outcomes. FedScore fills a gap in existing medical machine learning applications that lack established methods for generalizing unified scores across multiple sites. It also addresses the deficiency of the absence of reproducible benchmark methods, especially for more interpretable models.

The embodiments generate scoring models that are more interpretable due to the preference of using fewer variables and a more simplified model structure when compared with black box techniques such as deep learning based models. Conventional deep learning techniques also require a larger volume of data to generate models that are more accurate. In contrast, the disclosed FedScore system allows multiple nodes/sites to work towards a federated scoring model without the need for sharing confidential data. Since each site may have access to a limited volume of data, if each site pursues a deep learning based model, the outcomes at each site may not be optimal because of the limited volume of data. The FedScore system address this challenge by providing a more interpretable scoring model and generating a federated scoring model that addresses the constraints associated with black box machine learning models such as deep learning models. In addition, the scoring models being trained on data from multiple sites are more generalizable because of the diversity in the origin of data that such models as a whole are trained on. The federated scoring models generated by the embodiments are therefore more generalizable to new setting such as data from a new clinical setting or data from a different population. The disclosed FedScore framework could serve as the foundation of a data science software platform, which deals with large-scale, multi-centre data analysis and risk scoring development.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Although this disclosure describes or illustrates particular embodiments as providing particular advantages, particular embodiments may provide none, some, or all of these advantages.

1. A system for federated scoring, the system comprising:
a plurality of nodes,
a communication network enabling communication between the plurality of nodes,
each node comprising at least one processor and a memory, the memory of each node comprising sensitive data and program code,
the sensitive data comprises a plurality of records;
wherein the program code is executable by the respective processor of each node of the plurality of nodes to:
generate a first set of scoring model coefficients based on sensitive data accessible to the respective nodes;
broadcast the first set of scoring model coefficients to rest of the nodes;
and wherein at least one of the nodes is configured to:
receive contributory intermediate statistics from the rest of the nodes;
and generate a federated scoring model based on the received contributory intermediate statistics and its respective first set of scoring model coefficients.

2. The system of claim 1, wherein each node is configured to generate a node specific scoring model based on the first set of scoring model coefficients.

3. The system of claim 2, wherein the system further comprises a central server, wherein the central server is configured to evaluate each of the node specific scoring models and the federated scoring model based on model parsimony statistics.

4. The system of claim 3, wherein each record comprises a plurality of variables and each node is further configured to:
determine a rank of each of the plurality of variables based on the relevance of each of the variables to a scoring result generated by the respective scoring model;
transmit the ranks of each of the plurality of variables to the central server;
wherein the central server is configured to:
define a global variable rank based on the ranks of the plurality of variables received from the plurality of nodes;
transmit the global variable rank to at least one of the plurality of nodes.

5. The system of claim 4, wherein the relevance of each of the variables to a scoring result is evaluated based on model parsimony statistics or model area under curve statistics.

6. The system of claim 4, wherein the federated scoring model is generated based on the global variable rank.

7. The system of claim 6, wherein the federated model is generated by incorporating variables above a threshold in the global variable rank.

8. The system of claim 4, wherein the nodes determine a rank of each of the plurality of variables using a random forest model.

9. The system of claim 4, wherein the central server defines the global variable rank by averaging the rank of the plurality of variables received from each of the plurality of nodes.

10. The system of claim 1, wherein the scoring models are implemented using any one of: linear classification models, logistic regression models, clinical decision support models.

11. The system of claim 1, wherein the each of the plurality of nodes is configured to transmit its node specific scoring model and scoring model performance data to the central server.

12. The system of claim 11, wherein the central server is configured to receive the federated model from at least one of the nodes.

13. The system of claim 10, wherein the central server is configured to transmit the federated model to at least a subset of the plurality of nodes.

14. The system of claim 1, wherein the variables comprise one or more continuous data variables, and each of the nodes is further configured to transform the continuous data variables into discrete variables.

15. The system of claim 1, wherein at least one of the nodes is configured to process new clinical data using the federated model to generate a score.

16. The system of claim 1, wherein the contributory intermediate statistics are computed by each respective node based on the sensitive data accessible to the respective nodes.

17. A method for federated scoring comprising:
providing a plurality of nodes, each node comprising:
at least one processor and a memory,
the memory of each node comprising sensitive data and program code the sensitive data comprises a plurality of records;
providing a communication network enabling communication between the plurality of nodes,
executing the program code by the respective processor of each of the plurality of nodes to:
generate a first set of scoring model coefficients based on sensitive data accessible to the respective nodes;
broadcast the first set of scoring model coefficients to rest of the nodes;
executing the program code by at least one of the nodes to:
receive contributory scoring intermediate statistics from the rest of the nodes;
and
generate a federated scoring model based on the received contributory scoring intermediate statistics and its respective first set of scoring model coefficients.

18. One or more non-transitory computer-readable storage media storing instructions that when executed by one or more processors cause the one or more processors to perform the method of claim 17.
